(19) United States
(12) Patent Application Publication
(10) Pub. No.: US 2011/0195501 A1
(43) Pub. Date: Aug. 11, 2011

(54) ULTRASONICALLY INDUCED RELEASE FROM POLYMER VESICLES

(76) Inventors: Gautam D. Pangu, Philadelphia, PA (US); Daniel A. Hammer, Villanova, PA (US)

(21) Appl. No.: 13/057,238

(22) PCT Filed: Aug. 4, 2009
(86) PCT No.: PCT/US2009/052665

§ 371 Date: Apr. 28, 2011

Related U.S. Application Data
(60) Provisional application No. 61/086,518, filed on Aug. 6, 2008.

(51) Int. Cl. C12N 5/02

(52) U.S. Cl. 435/375

(57) ABSTRACT

Disclosed are methods of controllably permeabilizing polymersomes. Such methods are useful in permeabilizing polymersomes so as to effect controlled release of therapeutic or imaging agents to a particular location. Also disclosed are systems for controllably delivering various agents to particular locations via polymersomes and related polymersome-based methods for treating diseases and for imaging.

### RELATED APPLICATIONS

The present application claims the benefit of U.S. Pat. App. Ser. No. 61/086,518 (filed Aug. 6, 2008), the entirety of which application is incorporated herein by reference.

### STATEMENT OF GOVERNMENT INTEREST

The Government may have certain rights in the present invention. Research related to the present invention was supported by National Science Foundation grant NSF Penn 05-20020.

### FIELD OF THE INVENTION

The present invention relates to the fields of polymersomes and to controlled drug delivery.

### BACKGROUND OF THE INVENTION

Polymerosomes, otherwise known as polymer vesicles, are vesicles made from amphiphlic synthetic block copolymers. In aqueous solution, polymers of appropriate concentrations spontaneously self-assemble into vesicles with sizes from tens of nanometers to tens of microns.

These vesicles have a core-shell structure with separate hydrophilic and hydrophobic compartments and they exhibit several superior material properties over liposomes (vesicles comprised of natural phospholipids) and small molecule surfactant micelles. For example, polymerosome membranes are significantly thicker (ca. 9-22 nm) than those of liposomes (ca. 3-4 nm) which imparts significant mechanical strength and area strain to polymersomes. Polymersomes are also known to have chemical stability, biocompatibility and long in vivo circulation times.

In order to achieve efficient targeted drug delivery, a drug would ideally be stored stably in a container and be released as and when required. Challenges exist, however, in releasing stored drugs at the optimal location and in performing the release at the optimal time. Accordingly, there is a need in the art for systems and methods for controllably delivering drugs to the optimal location in the body under moderate conditions with minimal invasiveness to the patient.

### SUMMARY OF THE INVENTION

In meeting the described challenges, the present invention first provides methods of controllably permeabilizing a polymersome, comprising applying to at least one polymersome comprising a copolymer bilayer structure at least one cycle of sonic energy in the range of from about 20 kHz to about 5000 kHz, the at least one cycle of sonication giving rise to transient permeability of the polymersome.

Also provided are methods of controllably delivering an agent from a polymersome, the methods comprising subjecting at least one polymersome comprising a copolymer bilayer and one or more agents to one or more cycles of sonic energy to give rise to the release of one or more agents from the polymersome.

The present invention also describes systems for controllably delivering one or more agents, the systems comprising at least one polymersome comprising a bilayer outer portion and an inner portion contained by the bilayer outer portion, the polymersome further comprising an agent disposed within the inner portion, within the bilayer outer portion, or both; and a source of sonic energy, the source of sonic energy being capable of applying a frequency of at least 20 kHz, and wherein actuation of the source of sonic energy gives rise to release of the agent from the polymersome.

Also disclosed are methods of treating a disease, comprising introducing one or more polymersomes to a patient, the one or more polymersomes comprising bilayer structure and one or more therapeutic agents, applying sonic energy to the one or more polymersomes so as to effect controlled release of the one or more agents into the patient.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 depicts a schematic of the experimental set-up used to test the inventive polymersomes and methods;

FIG. 2 illustrates the change in fluorescence intensity with time for a OB29 polymer vesicle sample (a) without exposure to ultrasound where fluorescence reading was taken after every 20 minutes; and (b) after exposure to ultrasound, where ultrasound was applied in 20 min intervals with a 5 min gap between successive intervals for fluorescence measurements;

FIG. 3 illustrates the size distribution of OB29 vesicle samples (a) before sonication and (b) after sonication—the vesicle population shows more polydispersity after sonication and the average size increase;

FIG. 4 illustrates CTEM images of OB29 samples (a) and (b) before sonication and correspondingly (c) and (d) after sonication;

FIG. 5 illustrates the fluorescence intensity of an OB29 vesicle sample (a) after one sonication cycle (b) after two sonication cycles (c) after leaving the sample overnight at room temperature and (d) after third sonication cycle the next day. The intensity does not change significantly between (b) and (c) indicating that pores formed in vesicle membrane due to ultrasound are temporary and they reseal themselves after termination of ultrasound;

FIG. 6 illustrates the percentage ANTS release as a function of time for (a) OB29 polymer vesicle sample and (b) OB 18 polymer vesicle sample;

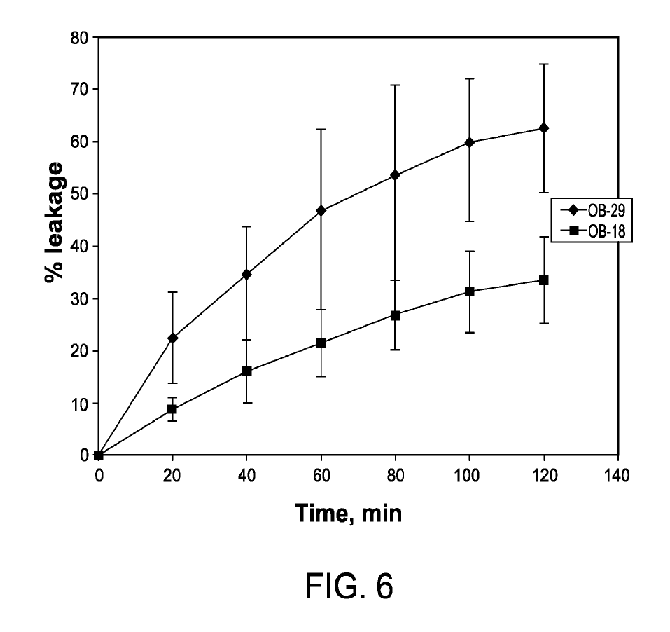

FIG. 7 illustrates the percentage ANTS release as a function of time for OB29 samples where (a) sonication interval of 10 min was used and (b) sonication interval of 20 min was used; and

FIG. 8 illustrates the percentage ANTS release as a function of time for OB29 samples where the average sonication power as recorded on dismembrator dial was (a) 2.5 W, (b) 3.5 W and (c) 5.5 W.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The present invention first provides methods of controllably permeabilizing a polymersome. These methods include applying to the at least one polymersome comprising a copolymer bilayer structure at least one cycle of sonic energy in the range of from about 20 kHz to about 5000 kHz, the at least one cycle of sonication giving rise to transient permeability of the polymersome. Application of multiple energy cycles is also within the scope of the claimed invention.

The at least one cycle of sonic energy suitably gives rise to one or more transient pores in the at least one polymersome. The methods suitably give rise to such pores without popping or otherwise disrupting the structure of the polymersomes.

The cycles of sonic energy are suitably applied in the power range of from about 0.01 W/cm² to about 10 W/cm², although the energy may be between about 0.1 W/cm² to about 5 W/cm², or even from about 0.5 W/cm² to about 1 W/cm². The optimal intensity will depend on the polymersomes being used and the user's desired release characteristics.

A cycle of sonic energy may last from about 1 minutes to about 60 minutes, or even from about 10 minutes to about 30 minutes. Cycles may even last from about 15 to about 25 minutes.

Polymersomes are suitably subjected to from 2 to 10 cycles of sonication, although from 3 to 5 cycles of sonication may also be suitable, depending on the needs of the user. The optimal frequency, power and number of cycles of sonic energy will be apparent to those of ordinary skill in the art, and can be determined without undue experimentation.

In some embodiments, the energy cycles are of the same frequency, the same power, the same duration, or all of these. In other embodiments, two or more of the sonic energy cycles are at a different power, a different frequency, a different duration, or all of these. Application of different frequencies and intensities of sonic energy enables the user to achieve a particular release pattern according to their needs.

Sonic energy suitable for the present invention is suitably in the range of from about 100 kHz to about 1000 kHz, or in the range of from about 500 kHz to about 750 kHz. In some embodiments, the frequency of the sonic energy has a frequency of from about 5 kHz to about 35 kHz. The cycles of sonic energy may be periodic, transient, or nonperiodic, and the optimal characteristics will be apparent to the user of ordinary skill in the field. The application of the sonic energy may be modulated by the user in response to a signal (e.g., a signal that indicates release of one or more agents from the polymersomes), or may be automated or otherwise computer-controlled.

The transient permeability of the polymersome suitably gives rise to release of an agent from the polymersome—as described, the polymersome is suitably provided to a cell, a biological tissue, an organ, a vessel, and the like. The polymersomes may be provided by implantation, injection, ingestion, or by other methods known in the art. Without being bound to the theory, the applied sonic energy gives rise to the formation of transient pores or openings in the sonicated polymersome.

Transient permeability of the polymersome is suitably reduced upon termination of the sonication. In some embodiments, the at least one cycle of sonic energy effects cavitation in a fluid phase contacting at least one polymersome.

As described elsewhere herein, the polymersome is permeabilized on account of the sonication. The sonication suitably permeabilizes the polymersome with little to no rupture, popping or other permanent disruption of the polymersome. Without being bound to any single theory, it is believed that the polymersome returns to or close to its normal state once the applied sonic energy is terminated. This enables the user to, by controlled application of sonic energy, effect multiple release events from the same polymersome over a period of time.

The permeabilization is used to deliver one or more contents of the polymersome to a particular location, as described elsewhere herein. The contents of the polymersome include, e.g., therapeutic or imaging agents. Dyes, chemotherapy agents, analgesics, radioactive labels, and the like may all be contained within a polymersome for subsequent release.

The present invention also includes methods of controllably delivering an agent from a polymersome. These methods include subjecting a polymersome residing at a location comprising a copolymer bilayer and one or more agents to one or more cycles of sonic energy to give rise to the release of one or more agents from the polymersome. The polymersome subjected to the present methods is suitably located at a location of interest to the user, such as a disease site or at a location about which the user seeks additional information.

As described elsewhere herein, the one or more cycles of sonic energy suitably gives rise to formation of one or more pores in the at least one polymersome. The sonic energy may be applied continuously, but may also be applied at random intervals or at periodic intervals, as described elsewhere herein.

Also as described elsewhere herein, the sonic energy is suitably of a frequency between about 20 kHz and about 5000 kHz, or in the range of from about 100 kHz to about 1000 kHz, or even in the range of from about 500 kHz to about 750 kHz. The sonic energy is suitably of a power between about 0.01 W/cm² and about 10 W/cm², or between about 0.1 W/cm² and about 5 W/cm², or even between about 1 W/cm² and about 3 W/cm². Various commercially-available sources of sonic energy are capable of supplying sonic energy of this energy level.

The disclosed methods also include scanning the at least one location to obtain one or more images of that location, and, where suitable, images of the polymersome at that location. The scanning may be optical, infrared, ultraviolet, x-ray, magnetic, electronic, or some other method known in the art, and may include application of sonic energy, radiation, magnetic field, acoustic energy, and the like. In some embodiments, the polymersome includes a dye, label, or other identifier to assist in locating the polymersome within a patient or to help determine the polymersome's location. In other embodiments, the polymersome is itself intrinsically visible to a scanning device. The scanning device may be capable of exciting the polymersome or its contents to provide information about the polymersome's location or may be capable of receiving information, such as a magnetic, optical, electromagnetic, or radioactive signal from the polymersome or its contents. This enables the user to track a polymersome in real-time, and to adjust the application of sonic energy accordingly.

Also disclosed are systems for controllably delivering one or more agents. The disclosed systems include at least one polymersome comprising a bilayer outer portion and an inner portion contained by the bilayer outer portion, the polymersome further comprising an agent disposed within the inner portion, within the bilayer outer portion, or both.

The systems also suitably include a source of sonic energy, the source of sonic energy being capable of applying a frequency of at least 20 kHz, and wherein actuation of the source of sonic energy—and application of sonic energy to the polymersome—gives rise to release of the agent from the polymersome.

The polymersomes of the present invention include one or more copolymer. Suitable copolymers include an alternating copolymer, a periodic copolymer, a random copolymer, a statistical copolymer, a diblock copolymer, a triblock copolymer, a branched copolymer, a star copolymer, a brush copolymer, a comb copolymers, a terpolymer, a graft copolymer, and the like. In some embodiments, the copolymer is blended with a phospholipid.

The copolymer also suitably includes a hydrophobic block and a hydrophilic block; polyethylene oxide-polybutadiene, polycaprolactone, and polylactic acid are all considered suitably copolymers. Polyethylene oxide attached to a hydrophobic polymer is also considered suitable for the polymersomes of the present invention, and suitable hydrophobic polymers will be apparent to those of ordinary skill in the art.

PEO-PBD copolymers are, as described elsewhere herein, considered especially suitable. The size of the PEO and PBD blocks in the copolymers may be varied according to the needs of the user. As described elsewhere herein, the physical properties of a polymersome may be tailored by altering the blocks of the copolymer.

The polymersome suitably includes a characteristic cross-sectional dimension in the range of from about 50 nm to about 10,000 nm, or in the range of from about 500 nm to about 5,000 nm, or even in the range of from about 1000 nm to about 3,000 nm. The polymersomes may be spherical, but they may also be ovoid or irregular in shape as well.

As described, the inventive polymersomes may include one or more therapeutic agents, and a variety of therapeutic agents are considered suitable for the present invention. A non-exclusive listing of such agents includes a therapeutic compounds, a particle, a nanoparticle, an acid, a base, a contrast agent, a dye, a fluorophore, an imaging agent, a contrast agent, a ligand, an oligonucleotide, a monomer, a polymer, a magnetic entity, a radioactive entity, a vitamin, DNA, RNA, a protein, a peptide, a drug, and the like. Chemotherapy agents are considered especially suitable for inclusion in the present invention.

In some embodiments, the polymer contains a fluid. In some embodiments, the polymersomes are suitably substantially free of air, gas, or any gaseous precursors.

A variety of devices may serve as sources of sonic energy in the present invention. Hydrophones, transducers, amplifiers, speakers, ultrasound probes, and the like are all suitable. Other sources of sonic energy will be known to those of ordinary skill in the art. In some embodiments, the source of sonic energy is capable of applying sonic energy to one or more locations, and may be capable of applying sonic energy to multiple locations simultaneously or of focusing sonic energy at one or more locations.

The energy source may apply sonic energy at periodic intervals, at random intervals, or both. The source of sonic energy is suitably capable of delivering sonic energy of at least about 0.01 W/cm².

The systems also suitably include an analysis device, capable of resolving the spatial location of the at least one polymersome. Such devices may be imaging devices or other similar equipment. The analysis device may, in some embodiments, determine the release of an agent from a polymersome or the behavior of such an agent once released. In some embodiments, the analysis device and source of sonic energy may be in electronic communication with one another—or even be the same device—so as to enable application of sonic energy based on data obtained by the analysis device.

Polymersomes according to the present systems may reside, at least initially, in a carrier medium. Water, buffers, biocompatible fluids, blood, plasma, biological fluids, preservatives, and the like are all considered suitable carrier media. Biocompatible carrier media are considered especially suitable, so as to facilitate safe introduction of the polymersomes to a subject.

The systems also include multiple polymersomes. In such embodiments, the polymersomes may be substantially identical in size, chemical composition, and physical structure to one another, or differ from one another in one or more of these respects. For certain applications, it may be advantageous to provide a population of polymersomes comprising polymers of different size or having different structural characteristics. The polymersomes of the present systems may contain the same or different therapeutic or imaging agents.

In some embodiments, the systems are used for imaging purposes. In such cases, the polymersomes may include a dye or other label that is detected by an imaging device. The dye or label may be visible to the imaging device while the dye or label resides within the polymersome or may become visible upon application of sonic—or other—energy to the polymersome. In some embodiments, a polymersome may include both imaging and therapeutic agents, with either type of agent being contained within the polymersome or being integrated, if suitable to the user's needs, into the polymersome membrane material. As described elsewhere herein, the imaging may include receiving a signal caused by exciting the polymersome or its contents, or both, or by receiving a signal that is inherently emitted by the polymersome, its contents, or both.

Also provided are methods of treating a disease. These methods include introducing one or more polymersomes to a patient, the one or more polymersomes comprising bilayer structure and one or more therapeutic agents, and applying sonic energy to the one or more polymersomes so as to effect controlled release of the one or more agents into the patient.

The application of the sonic energy suitably, as described elsewhere herein, effects the formation of one or more transient pores in the one or more polymersomes, which polymersomes remain substantially unruptured as a result of the application of the sonic energy.

The therapeutic agents may be selected based on the disease being treated. For example, a chemotherapy agent may be chosen if the patent suffers from cancer.

In some embodiments, the one or more polymersomes binds specifically to a cell, tissue, organ, organelle, or other biological entity. This binding enable precise delivery of the agent or agents of the polymersome to a particular location. Agents for such delivery may be contained within the polymersome, but may also be incorporated into the polymersome itself or onto the outside of the polymersome.

The polymersome binding is suitably mediated by a peptide, an antibody, a carbohydrate, or other molecule or biological entity capable of specific, targeting binding. Ligand/receptor interactions are considered suitable for such binding, as are peptides, antibodies, carbohydrates, and the like. The polymersomes may incorporate any number of molecules capable of specific binding to complementary biomarkers displayed on the surface of cells or other bodily tissues. The net result of such binding is that polymersomes may act as disease-seeking delivery vehicles.

The methods may also include imaging the one or more polymersomes. This may be accomplished by application of sonic energy, radiation, magnetic field, acoustic energy, and the like, as described elsewhere herein. The imaging may be accomplished live in some embodiments or at intervals. In one sample embodiment, a polymersome containg a therapeutic agent binds specifically to a disease site. Once the polymersome is bound to the disease site, the polymersome is sonicated so as to release its therapeutic contents directly at the disease site. In some cases, the location of the polymersome is monitored before sonication so as to confirm that the polymersome has bound to the disease site before sonication.

In some embodiments, the polymersomes may be used to detect the presence of a disease and treat that disease. In these embodiments, the polymersome is configured such that it binds specifically to one or more biological markers that indicate a disease. The polymersome's binding—and, hence, location—are monitored, and the binding of the polymersome then indicates the presence of a disease. The polymersome may then be sonicated to release its therapeutic agent or agents at the disease site. Such polymersomes enable integrated disease detection and treatment.

### EXAMPLES AND NON-LIMITING EMBODIMENTS

Several exemplary, non-limiting embodiments of the disclosed invention were evaluated, as set forth below.

Materials: Polyethylene oxide-Polybutadiene diblock copolymers used in this work, OB-29 (PEO₂₆—PBD₄₆, M.W. 3800 Da) and OB-18 (PEO₈₀—PBD₁₂₅ M.W. 10400) were purchased from Polymer Source (Dorval, PQ, Canada). 8-aminonaphthalene-1,3,6-trisulfonic acid, disodium salt (ANTS), and p-xylene-bis-pyridinium bromide (DPX) were obtained from Molecular Probes (Eugene, Oreg.). Phosphate buffered saline (PBS) tablets (pH 7.4), methylene chloride (CH₂Cl₂), sodium chloride (NaCl), calcium chloride (CaCl₂), sodium azide NaN₃), 4-(2-hydroxyethyl)-1-piperazineethanesulfonic acid (HEPES) and Triton X-100 were purchased from Sigma-Aldrich (St. Louis, Mo.). Slide-A-Lyzer dialysis cassette kit (20K molecular weight cutoff) was purchased from Pierce (Rockford, Ill.). All materials were used as received.

Preparation of leakage buffer: to measure vesicle permeation by ultrasound, a leakage buffer developed by Ellens et. al. was used. That buffer contained 60 mM NaCl, 5 mM CaCl2, 5 mM HEPES, 3 mM Nd3, 12.5 mM ANTS and 45 mM DPX and after preparation, it was equilibrated to a pH of 7.4. When encapsulated within the core of the vesicles, DPX quenches ANTS fluorescence due to molecular proximity. However, when ANTS and DPX are released in the bulk, the average distance between the molecules increases and a sample shows increased fluorescence. During fluorescence measurements, the excitation wavelength applied to the ANTS buffer was 355 nm, and the emission spectra were recorded for wavelengths ranging from 450 nm to 550 nm.

Formation of polymer vesicles: Small polymer vesicles were prepared via the following procedures. Briefly, a thin film of polymer was deposited on the bottom of a glass vial by overnight evaporation of 200 μl of 1 mM solution of the polymer in CH₂Cl₂ under vacuum. Polymerosomes were formed by hydration with 2 ml of ANTS-DPX buffer and sonication of the sample at 60° C. for 60 min in a bath sonicator (model FS 20; Fisher Scientific, PA). The vesicles were extruded to a suspension with narrow size distribution using a Liposofast Basic hand-held extruder equipped with a 100 nm polycarbonate membrane (Avestin Inc., Ottawa, Ontario). Excess buffer in the external solution was removed by dialysis using 20K MW cutoff dialysis cassettes.

Vesicle samples were injected into the dialysis cassette with a syringe, and the cassette was immersed in a beaker filled with iso-osmatic PBS solution (Advance Instruments, Norwood, Mass., osmometer model 3300). PBS buffer was replaced every day for three days with fresh iso-osmotic PBS and vesicles were subsequently removed from dialysis. Vesicle size distribution was measured by using zetasizer nano S90 (Malvern Instruments, Worcestershire, UK). Quartz cuvettes were filled with 1 ml of polymersome suspensions and were thermostatically controlled at 25° C. throughout the experiment. All DLS measurements were made at a scattering angle of 90″. The results are given as percent intensity as a function of size.

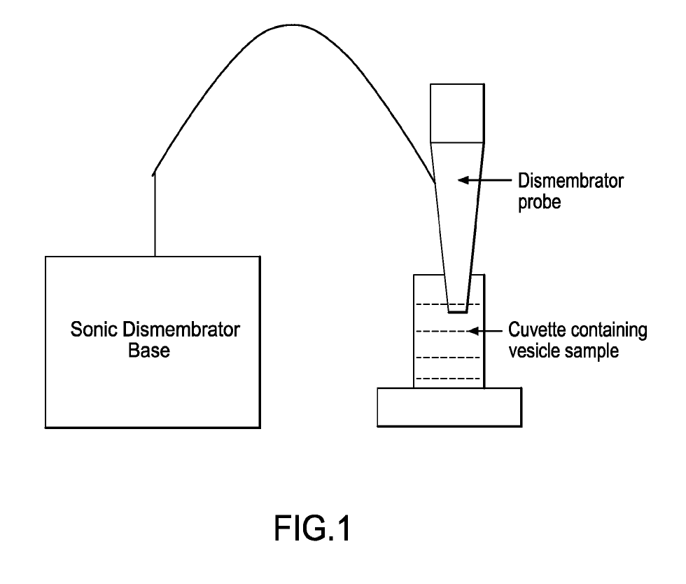

Sonication and fluorescence measurements: Sonication measurements in this work were carried out using a commercial 20 KHz sonic dismembrator (model 100, Fisher Scientific, Pittsburgh, Pa.) with a 3 mm diameter microtip. The microtip was immersed in a 1.5 ml polystyrene cuvette (10 mm path length), containing 1 ml vesicle sample up to a marked position on the cuvette (1 cm from the bottom surface of the cuvette). The schematic of the set up is shown in FIG. 1. One sonication cycle consisted of sonication for a set duration of time (20 min unless otherwise stated) followed by a 5 min gap during which the ultrasonic exposure was terminated, the cuvette was removed from the set up and a fluorescence measurement was performed on the sample. The fluorescence measurements were carried out using a fluorolog-3 steady state spectrofluorometer (Horiba Jobin Yuon, Edison, N.J.).

The excitation wavelength applied to ANTS buffer was 355 nm and the emission spectra was recorded for wavelengths ranging from 450 nm to 550 nm. The leakage of ANTS from vesicle core was quantified by using surfactant mediated destabilization of vesicle membrane. A small surfactant molecule like, e.g., Triton X-100 can induce catastrophic membrane destabilization due to its rapid partitioning in and out of the membrane. Small volume (20-30 μl) of 20% Triton X-100 solution was added to vesicle sample after completion of all sonication cycles and the fluorescence was measured. Percentage leakage for each of the sonication cycles was then calculated by using the relation:

% leakage=(F_{cycle}−Fᵢₙᵢₜᵢₐₗ)/(F_{final}−Fᵢₙᵢₜᵢₐₗ)

where F_{final} is the fluorescence recorded after addition of Triton X-100, F_{cycle} is the fluorescence recorded after a particular sonication cycle and Fᵢₙᵢₜᵢₐₗ is the fluorescence before sonication. For consistency, fluorescence recorded at 520 nm was used in all the calculations. Size distribution of vesicle sample was measured before causing complete rupture with Triton X-100.

Cryogenic Transmission Electron Microscopy (CTEM): Vitreous samples for CTEM were prepared within a controlled environment vitrification system (CEVS). A droplet of solution (˜10 μl) was deposited on a polymer-carbon film coated copper TEM grid. A thin film (10-300 nm) was obtained by blotting with filter paper. The grid was then plunge cooled in liquid ethane at its freezing point (˜90K), resulting in vitrification of the aqueous film. Sample grids were examined in a JEOL 1210 transmission electron microscope operating at 120 kV, and images were recorded with a Gatan 724 multiscan digital camera.

Results:

Characterization of Vesicle Response to Ultrasound

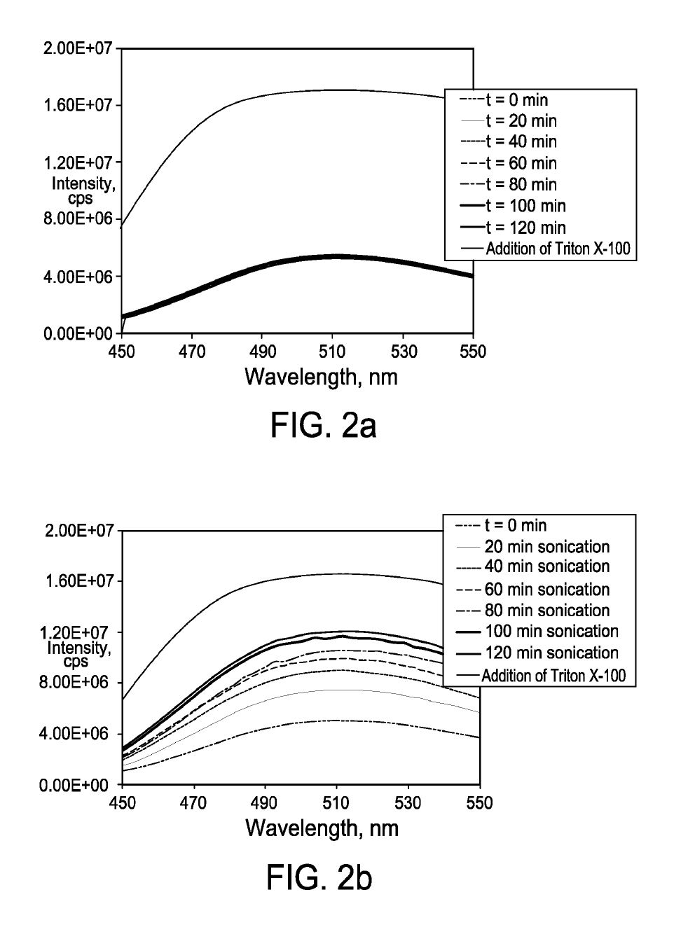

The representative results for fluorescence spectra of OB-29 polymer vesicle samples with and without ultrasonic exposure are shown in FIG. 2. The power level applied to the sample was 40% of full power of the model 100 dismembrator (corresponding to an average power reading of 5.5 W on the dismembrator controller) unless otherwise stated. The negative control samples were not subjected to ultrasound and fluorescence readings of the samples were taken after every 20 minutes.

Initially, none of the samples show a fluorescence peak around 520 nm, which may show that quenching of ANTS fluorescence by DPX was mostly complete. The negative control samples did not show appreciable change in fluorescence at 520 nm over time, which likely meant that leakage of ANTS from aqueous vesicle core was negligible without ultrasonic exposure. Samples subjected to ultrasound showed significant increase in fluorescence around 520 nm after just one sonication cycle, and generally fluorescence intensities increased with additional sonication cycles before approaching a steady value. Without being bound to any one theory, the increase in fluorescence was apparently due to ANTS leaking out of the vesicle cores in the bulk solution.

The effects of ultrasound on membrane-bound structures are generally associated with stable or transient cavitation. Based on the intensity of interaction between a membrane and a cavitation bubble, ultrasound may completely tear off a vesicle membrane resulting in several smaller vesicular or micellar structures. Otherwise, the effects of ultrasound on vesicle membrane may result in temporary pore formation in vesicle membrane which may or may not reseal after ultrasonic exposure is terminated.

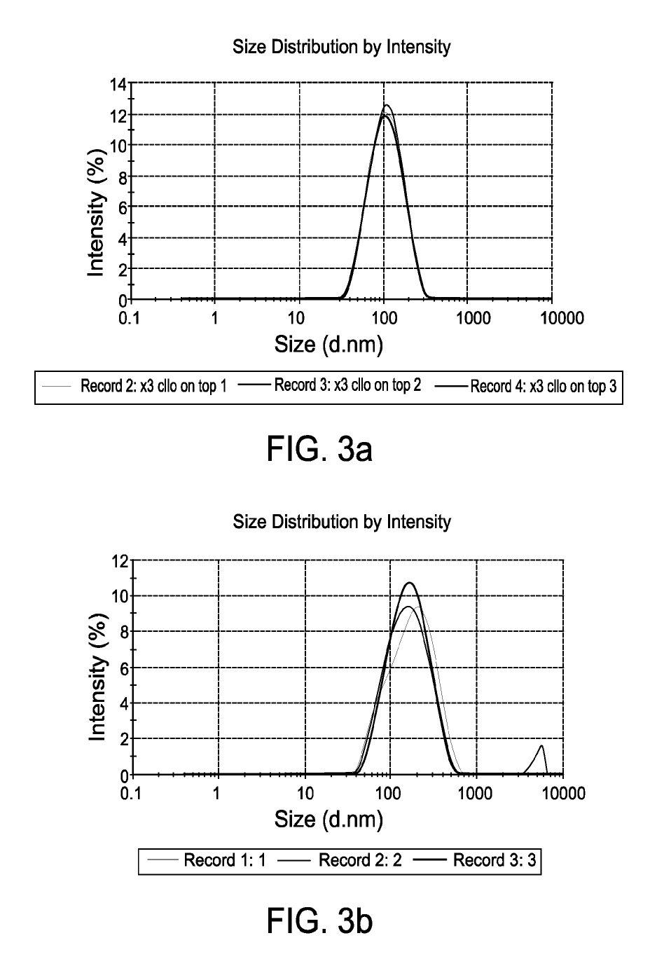

To understand the effect of ultrasound on polymer vesicles, size distribution measurement and cryogenic transmission electron microscopy (CTEM) were performed on vesicle populations before and after sonication. FIG. 3 shows typical size distribution results for OB-29 vesicles before and after 6 sonication cycles of 20 minutes duration each. The average diameter for all vesicle samples before sonication was 100+17 nm. Generally, the sonicated samples showed more 'polydispersity than the original sample as indicated by 3 different locations of size distribution peaks from 3 different measurements on sonicated samples in FIG. 3(b). However for all the samples studied, the size distributions did not change significantly after sonication. Noticeably, size distribution after sonication did not show any significant presence of structures smaller than the vesicles in the original sample. Without being bound to any single theory, if ultrasound were breaking the vesicles into smaller structures, the size distribution after sonication would likely have been bimodal, showing a sizable fraction of population at a smaller size as observed by Lin and Thomas, Langmuir 19 (2003), 1098-1105, for insonation of liposomes containing polyethylene glycol.

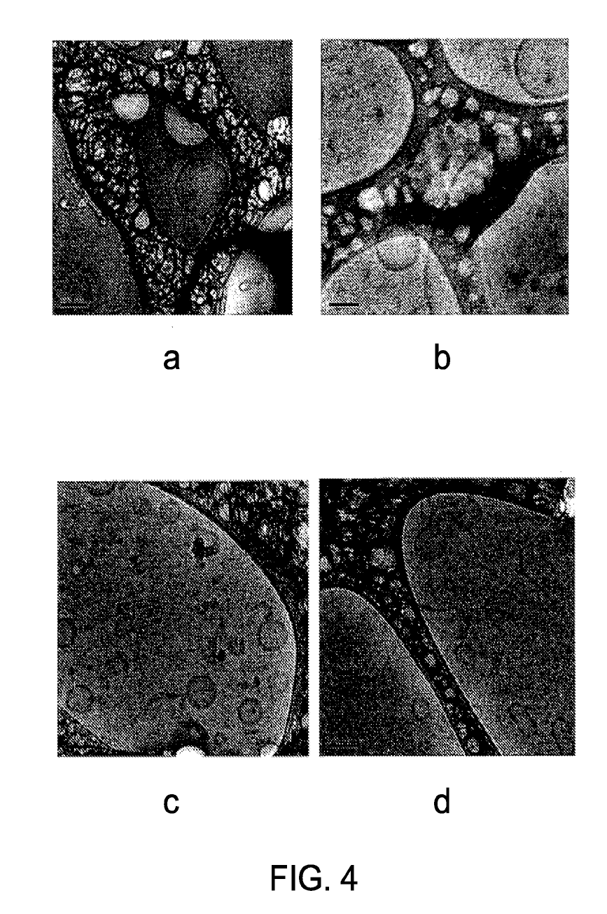

Two sets of CTEM images of vesicles before and after sonication are shown in FIG. 4. All CTEM images of vesicle samples after sonication revealed the presence of intact vesicles. FIG. 4(a) and (b) show vesicles of around the same size in the sample before and after sonication respectively. FIG. 4(c) and (d) (before and after sonication respectively) also show the vesicles in the same size range.

However, the image in FIG. 4(d) contains proportionally more non-spherical vesicles than the image in FIG. 4(c). This loss of sphericity may, without being bound to the theory, be the result of repeated exposure of vesicles to cavitation bubbles which, based on the type of cavitation, release large amount of pressure as they collapse (transient cavitation) or exert shear stress on vesicle membrane due to oscillatory flows (stable cavitation). This repeated exposure may have resulted in decrease in tension and rigidity of vesicle membrane resulting in a more stretched bilayer. This may explain the observed increase in polydispersity of vesicle population after sonication to some extent. These results suggest that rather than completely breaking apart, polymer vesicle membrane undergoes localized pore formation as a result of ultrasonic exposure.

To determine if ultrasonic exposure causes permanent pores in vesicle membranes, leakage of ANTS from vesicles was monitored with time after ultrasound was terminated. FIG. 4 shows the results of one such experiment where OB-29 vesicles were subjected to one and two sonication cycles (graphs a and b respectively) and then stored overnight at room temperature. The fluorescence intensity was measured the next day (graph c) and it did not show significant change overnight. However one sonication cycle later, the fluorescence increased again (graph d). This trend was quite reproducible, and indicated that ANTS did not leak out of the vesicle cores once ultrasonic exposure was terminated. The leakage occurs only during the exposure to ultrasound. This also indicates that ANTS that was leaked out in first two sonication cycles did not circulate back into the aqueous vesicle cores overnight as that would have resulted in requenching of ANTS fluorescence. These measurements suggest that ultrasonic exposure forms temporary pores in vesicle membrane which reseal after exposure is terminated.

Polymer vesicles are known for their toughness and high elastic and bending moduli. Also, vesicles with average size of 100 nm were used in this work. With decreasing size, pressures of greater magnitude would be required to rupture vesicle membrane. Hence, it is not surprising that these membranes can withstand high local pressures and temperatures or high shear stresses in the vicinity of cavitating bubbles.

Next, quantifying ANTS leakage and its dependence on membrane composition and physical parameters of sonication was explored.

Dependence of ANTS Leakage on Polymer Properties and Sonication Parameters

As mentioned elsewhere herein, the % leakage of ANTS from vesicles can be quantified by comparison to leakage caused by catastrophic destabilization of vesicle membrane by Triton-X 100.

To study the effect of membrane properties on leakage, the ultrasonic response of polymer vesicles made from two polymers, OB-29 and OB-18 respectively was explored. OB-18 (PEO₈₀—PBD₁₂₅) has both a larger PEO head group and a longer PBD tail as compared to OB-29 (PEO₂₆—PBD₄₆) The average membrane thickness of polymersomes synthesized from OB-29 and OB-18 has been estimated by cryoTEM to be 8 nm and 14 nm, respectively. Without being bound to any single theory, it is probable that the larger PEO head groups of OB-18 may form larger pores or assist in stabilizing the pores and facilitate increased leakage from aqueous core. However, it is also likely that the thicker bilayer of OB-18 may offer increased resistance to ultrasonic destabilization of membrane.

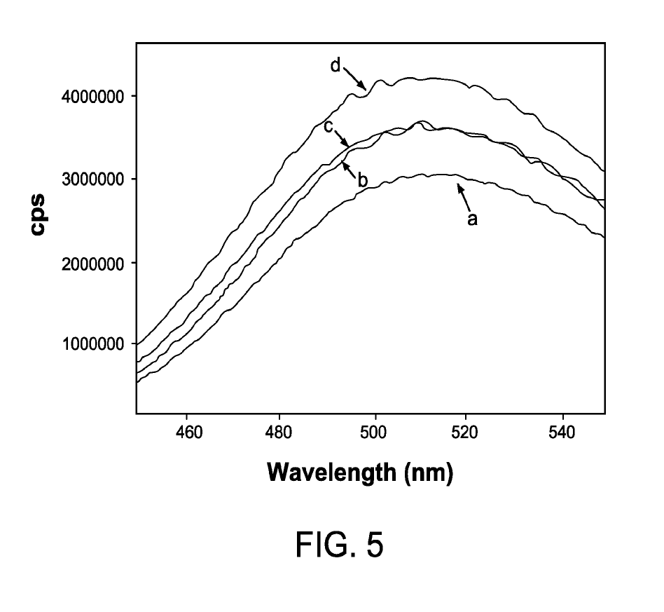

The average size of OB-18 vesicles was 107±20 nm, which is very similar to the average size of OB-29 vesicles (100±17 nm). The results showing percentage leakage of ANTS with time for OB-18 and OB-29 polymer vesicles are shown in FIG. 5. All vesicle samples were subjected to 6 sonication cycles each of 20 min duration and then Triton X-100 was added for full release. Time value on the x-axis refers to the actual sonication time (20 min of ‘ultrasound on’ period per cycle) and does not take into account the 5 min gap between successive sonication cycles. OB-29 vesicles showed much significant leakage than OB-18 vesicles with the % leakage values around 60 after 6 sonication cycles of 20 min each. Without being bound to any single theory, this indicated that the thicker membrane plays a more important role than a bulkier headgroup in governing the amount of leakage. The kinetics of leakage is also slightly different for two vesicle samples. For OB-29, the initial leakage profile is higher (ca. 1.12% per minute), than for OB-18 vesicles (0.45% per minute), followed by a gradual increase in percentage leakage which eventually reaches a plateau after 6 sonication cycles. The leakage profile for OB-18 is more uniform over time. However even it seems to have reached a plateau after 6 sonication cycles. The thinner OB-29 membrane is likely to give rise to more and/or bigger pores, causing faster ANTS leakage during first cycle. However, for subsequent cycles the leakage is likely to have slowed down because of the lesser driving force for ANTS to leak into the bulk. However it is still substantially greater and faster than leakage from OB-18 vesicles at all times.

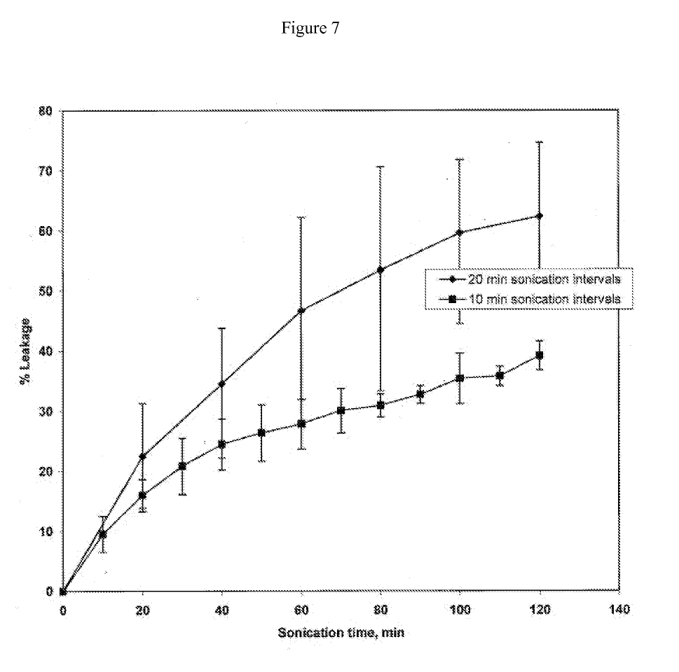

The effect of two sonication parameters—sonication time intervals and sonication intensity—were also studied. FIG. 7 shows the comparison of leakage profiles for 10 min and 20 min sonication intervals. In both cases, there was a 5 min gap between successive measurements for a fluorescence reading. The % leakage and leakage rate for 20 min intervals is clearly higher than that for 10 min intervals. Clearly, two 10 min intervals are not as effective as one 20 min interval in inducing leakage. Curiously, the % leakage for 10 min intervals reached a plateau after about 6 sonication intervals (time=60 min) even though it was only 27% at that time. This was probably due to lesser number of pores or smaller size of pores or a combination of both when 10 min interval was used. The process of membrane permeation is believed to be dependent on cavitation for which formation, growth and collapse of gas bubbles is essential. The longer sonication intervals allow sufficient time for these steps to occur, whereas shorter sonication intervals followed by 5 minutes of “ultrasound-off” periods may, depending on conditions, be insufficient for bubbles to form and grow by rectified diffusion to their resonance size. In some initial proof-of-principle experiments, a time interval of 5 min was used (data not shown) which resulted in no physical agitation in the mixture and negligible leakage from the vesicles. Thus, sonication time interval is one parameter that influences the leakage profiles.

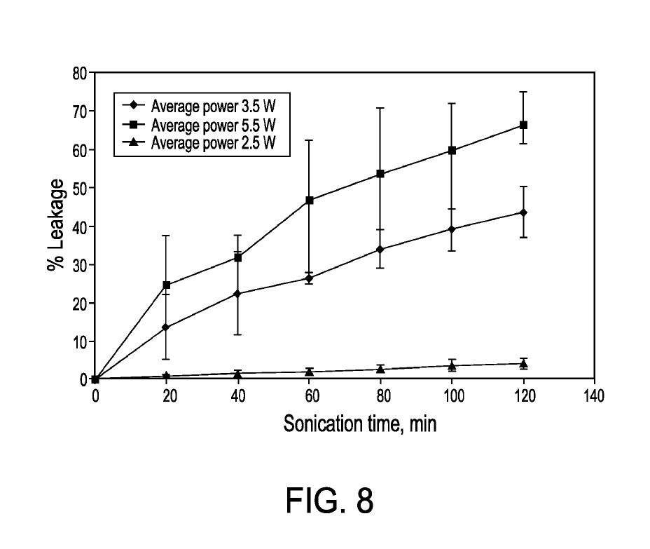

The effect of using different sonication intensities on ANTS leakage is shown in FIG. 8. Three intensities corresponding to 1o %, 20% and 40% of full dismembrator power were used. The average power readings on the dismembrator dial for these two settings during the experiments were 2.5 W, 3.5 W and 5.5 W respectively. The leakage was sensitive to amount of power applied with stronger ultrasonic fields causing greater leakage. When greater than 50% of full dismembrator power was used, violent agitation was observed in vesicle sample. The results show a clear threshold with respect to power as far as the ability of ultrasound to cause leakage is concerned. Without being bound to the theory, the leakage is assumed to be a cavitation-dependent process, and cavitation itself occurs only above a threshold acoustic power above which the peak pressure during rarefaction cycle drops below the tensile strength of the buffer. Thus, the leakage threshold observed in these results probably correlates with the cavitation threshold at 20 kHz. While these intensities are higher than some ultrasonic intensities used for diagnostic applications, they are within the therapeutic ultrasonic intensity range

### SUMMARY

In sum, an experimental system to study the response of polymer vesicles to low frequency ultrasound has been developed. Polymersome response to sonic energy was studied by measuring the leakage of a fluorescent dye from aqueous cores of polymer vesicles. Vesicles showed leakage above a certain ultrasonic power threshold, and without being bound to any single theory, this result suggests that leakage is a cavitation-mediated event. The leakage profiles showed a strong dependence on thickness of membrane bilayer of vesicles, sonication time and intensity. The dynamic light scattering measurements and cryogenic transmission electron microscopy on vesicle population indicated that insonation does not result in complete vesicle breakage but forms temporary pores in membranes which are resealed after ultrasound is terminated.

Polymer vesicles are tougher than liposomes. A comparison of the obtained results with the studies of ultrasonically induced leakage from liposomes reveals that the percentage leakage from polymer vesicles of similar sizes was comparable to that observed for liposomes. However, most of these studies used sonication intervals of 5 or 10 min for their experiments. Also, a sonication cycle followed by an “ultrasound-off” period for fluorescence reading may have had a different effect on formation and growth of cavities than concurrent sonication process and fluorescence measurement. The membrane composition was varied by forming vesicles from two different polymers with different head size and tail length. Another way to alter the membrane would be to incorporate different functional groups in head groups or tails of the copolymers, as the leakage process may be enhanced by incorporation of lipids with poly(ethylene glycol) groups or other species into the membrane. This may enable another way, in addition to changing head group size and tail length, to tailor membrane properties to control leakage.

The frequency of ultrasonic field is also a parameter that affects the technique from both physical and application point of view. While low frequency ultrasound was observed to induce significant leakage from vesicles, it may not always be convenient in biomedical applications. As the frequency goes up, the power threshold required to induce cavitation also increases. Hence at these frequencies, higher intensities may be required to induce significant leakage. When ultrasound of such high intensities is used in biomedical applications, damage to cells and tissues is always a concern. But this problem can be addressed by using focused transducers to generate and focus ultrasonic energy to a small region thereby minimizing the damage to surrounding cells and tissues. Further, ultrasonic contrast agents have been developed recently for diagnostic applications, and could serve as external cavitation sites. The power threshold for cavitation in presence of these agents is observed to be lesser than that without externally added gas bubbles. Thus, in the presence of these agents, drug delivery may be combined with diagnostic applications and can be achieved at lower acoustic intensities.

The disclosed invention is the first systematic study of response of polymer vesicles to physical forces exerted by an ultrasonic field. The strong dependence of membrane permeation on ultrasonic and chemical parameters suggests the possibility of tailoring them to get desired'extent of membrane destabilization. The results indicate that ultrasound has potential as a therapeutic tool for drug delivery from polymer vesicles.

Accordingly, this work assessed the effect of comparatively low frequency ultrasound (20 KHz) on polymer vesicles formed from PEO-PBD copolymers. The permiabilization of vesicles is measured by loading a fluorescent molecule in aqueous core of the vesicles with another molecule that quenches its fluorescence. Fluorescence is regained when the fluorophore is released from the core as a result of ultrasonic exposure. The ultrasonic effect on vesicle structure is characterized by performing dynamic light scattering measurements and cryogenic transmission electron microscopy on vesicle population before and after ultrasonic exposure in addition to fluorescence measurements. The results indicate that ultrasonic exposure does not result in complete vesicle lysis, but it does cause transient pore formation in vesicle membrane which results in leakage from aqueous core. The pores reseal after ultrasound is terminated. The rate and amount of leakage shows some dependence on membrane properties and physical parameters of ultrasound, and these properties may be tuned to achieve desired drug release profiles from polymer vesicles.

What is claimed:

1. A method of controllably permeabilizing a polymersome, comprising:
applying to the at least one polymersome comprising a copolymer bilayer structure at least one cycle of sonic energy in the range of from about 20 kHz to about 5000 kHz,
the at least one cycle of sonication giving rise to transient permeability of the polymersome.

2. The method of claim 1, wherein the at least one cycle of sonic energy gives rise to one or more transient pores in the at least one polymersome.

3. The method of claim 1, wherein the at least one cycle of sonic energy is applied in the power range of from about 0.01 W/cm² to about 10 W/cm².

4. The method of claim 1, wherein the at least one cycle of sonic energy is applied in the power range of from about 0.1 W/cm² to about 5 W/cm².

5. The method of claim 1, wherein the at least one cycle of sonic energy is applied in the power range of from about 0.5 W/cm² to about 1 W/cm².

6. The method of claim 1, wherein the at least one cycle of sonic energy lasts from about 1 minutes to about 60 minutes.

7. The method of claim 1, wherein the at least one cycle of sonic energy lasts from about 10 minutes to about 20 minutes.

8. The method of claim 1, wherein the at least one cycle of sonic energy effects cavitation in a fluid phase contacting at least one polymersome.

9. The method of claim 1, wherein the polymersome is subjected to from two to ten cycles of sonication.

10. The method of claim 1, wherein the polymersome is subjected to from 3 to 5 cycles of sonication.

11. The method of claim 9, wherein two or more of the sonic energy cycles have the same frequency, the same power, or both.

12. The method of claim 9, wherein two or more of the sonic energy cycles are at a different power, a different frequency, or both.

13. The method of claim 1, wherein the sonic energy is in the range of from about 100 kHz to about 1000 kHz.

14. The method of claim 1, wherein the sonic energy is in the range of from about 500 kHz to about 750 kHz.

15. The method of claim 9, wherein two or more of the cycles of sonic energy are periodic, nonperiodic, or any combination thereof.

16. The method of claim 1, wherein the transient permeability of the polymersome is reduced upon termination of the sonication.

17. The method of claim 1, wherein the transient permeability of the polymersome gives rise to release of an agent from the polymersome.

18. The method of claim 1, wherein the polymersome remains essentially unruptured as a result of the sonic energy.

19. The method of claim 1, wherein the polymersome is provided to a cell, a biological tissue, an organ, a vessel, or any combination thereof.

20. A method of controllably delivering an agent from a polymersome to a location, comprising:
subjecting at least one polymersome, residing at a location, comprising a copolymer bilayer and one or more agents to one or more cycles of sonic energy to give rise to the release of one or more agents from the polymersome.

21. The method of claim 20, wherein the one or more cycles of sonic energy gives rise to formation of one or more pores in the at least one polymersome.

22. The method of claim 20, wherein the sonic energy is applied continuously.

23. The method of claim 20, wherein the sonic energy is applied at random intervals, at periodic intervals, or any combination thereof.

24. The method of claim 20, wherein the sonic energy is of a frequency between about 20 kHz and about 5000 kHz.

25. The method of claim 1, wherein the sonic energy is in the range of from about 100 kHz to about 1000 kHz.

26. The method of claim 1, wherein the sonic energy is in the range of from about 500 kHz to about 750 kHz.

27. The method of claim 20, wherein the sonic energy is of a power between about 0.01 W/cm² and about 10 W/cm².

28. The method of claim 20, wherein the sonic energy is of a power between about 0.1 W/cm² and about 5 W/cm².

29. The method of claim 20, wherein the sonic energy is of a power between about 1 W/cm² and about 3 W/cm².

30. The method of claim 20, further comprising scanning the at least one location to obtain one or more images of that location.

31. The method of claim 30, wherein scanning comprises application of sonic energy, radiation, magnetic field, acoustic energy, or any combination thereof.

32. A system for controllably delivering one or more agents, comprising:
at least one polymersome comprising a bilayer outer portion and an inner portion contained by the bilayer outer portion,
the polymersome further comprising an agent disposed within the inner portion, within the bilayer outer portion, or both; and
a source of sonic energy,
the source of sonic energy being capable of applying a frequency of at least 20 kHz, and
wherein actuation of the source of sonic energy gives rise to release of the agent from the polymersome.

33. The system of claim 32, wherein the polymersome comprises a copolymer.

34. The system of claim 33, wherein the copolymer comprises an alternating copolymer, a periodic copolymer, a random copolymer, a statistical copolymer, a diblock copolymer, a triblock copolymer, a branched copolymer, a star copolymer, a brush copolymer, a comb copolymers, a terpolymer, a graft copolymer, or any combination thereof.

35. The system of claim 33, wherein the copolymer is blended with a phospholipid.

36. The system of claim 34, wherein the copolymer comprises a hydrophobic block and a hydrophilic block.

37. The system of claim 34, wherein the copolymer comprises polyethylene oxide-polybutadiene, polycaprolactone, polylactic acid, or polyethylene oxide attached to a hydrophobic polymer.

38. The system of claim 32, wherein the at least one polymersome comprises a characteristic cross-sectional dimension in the range of from about 50 nm to about 10,000 nm.

39. The system of claim 32, wherein the at least one polymersome comprises a characteristic cross-sectional dimension in the range of from about 500 nm to about 5,000 nm.

40. The system of claim 32, wherein the at least one polymersome comprises a characteristic cross-sectional dimension in the range of from about 1000 nm to about 3,000 nm.

41. The system of claim 32, wherein the agent comprises a therapeutic compound, a particle, a nanoparticle, an acid, a base, a contrast agent, a dye, a fluorophore, an imaging agent, a contrast agent, a ligand, an oligonucleotide, a monomer, a polymer, a magnetic entity, a radioactive entity, a vitamin, DNA, RNA, a protein, a peptide, or any combination thereof.

42. The system of claim 32, wherein the polymersome is substantially free of gas or any gaseous precursors.

43. The system of claim 32, wherein the source of sonic energy comprises a hydrophone, a transducer, an amplifier, a speaker, a probe, or any combination thereof.

44. The system of claim 32, wherein the source of sonic energy is capable of applying sonic energy to one or more locations.

45. The system of claim 44, wherein the source of sonic energy is capable of focusing sonic energy at one or more locations.

46. The system of claim 32, wherein the source of sonic energy is capable of applying sonic energy at periodic intervals, at random intervals, or any combination thereof.

47. The system of claim 32, further comprising an analysis device.

48. The system of claim 47, wherein the analysis device is capable of resolving the spatial location of the at least one polymersome.

49. The system of claim 32, wherein the source of sonic energy is capable of delivering sound at a power of at least 0.01 W/cm².

50. The system of claim 32, wherein the at least one polymersome initially resides in a carrier medium.

51. The system of claim 32, wherein the carrier medium comprises water, a buffer, a biocompatible fluid, blood, plasma, a biological fluid, a preservative, or any combination thereof.

52. The system of claim 32, wherein the system comprises a plurality of polymersomes.

53. The system of claim 32, wherein two or more of the plurality of polymersomes are substantially identical in size, chemical composition, and physical structure.

54. The system of claim 32, wherein two or more of the plurality of polymersomes differ in size, chemical composition, physical structure, or any combination thereof.

55. The system of claim 32, wherein the system is used for imaging.

56. The system of claim 32, wherein the system is used to treat one or more illnesses, diseases, or other medical conditions.

57. A method of treating a disease, comprising:
introducing one or more polymersomes to a patient,
the one or more polymersomes comprising bilayer structure and one or more therapeutic agents,
applying sonic energy to the one or more polymersomes so as to effect controlled release of the one or more agents into the patient.

58. The method of claim 57, wherein the sonic energy effects the formation of one or more transient pores in the one or more polymersomes.

59. The method of claim 57, wherein the one or more polymersomes remain substantially unruptured as a result of the application of the sonic energy.

60. The method of claim 57, wherein the one or more therapeutic agents is selected based on the disease.

61. The method of claim 57, wherein the one or more polymersomes binds specifically to a cell, tissue, organ, organelle, or other biological entity.

62. The method of claim 61, wherein the binding is mediated by a peptide, an antibody, a carbohydrate, or any combination thereof.

63. The method of claim 62, wherein the polymersome comprises a peptide, an antibody, a carbohydrate, or any combination thereof.

64. The method of claim 57, further comprising imaging the one or more polymersomes.

65. The method of claim 64, further comprising applying sonic energy to the one or more polymersomes in response to the results of the imaging.
